(19) United States
(12) Patent Application Publication
(10) Pub. No.: US 2003/0105376 A1
(43) Pub. Date: Jun. 5, 2003

(54) Purification of polyolefin feedstocks using multiple adsorbents

(76) Inventors: Michael J. Foral, Aurora, IL (US); Bruce D. Alexander, Lombard, IL (US); Larry C. Satek, St. Charles, IL (US); Brian C. Bahr, Batavia, IL (US)

Correspondence Address: CAROL WILSON, BP AMERICA INC., MAIL CODE 5 EAST, 4101 WINFIELD ROAD, WARRENVILLE, IL, 60555, US

(21) Appl. No.: 09/997,787

(22) Filed: Nov. 30, 2001

(51) Int. Cl. C07C 7/12; C07C 7/00

(52) U.S. Cl. 585/804

(57) ABSTRACT

Processes using heterogeneous adsorbents are disclosed for purification of olefins to obtain feedstocks suitable for formation of olefin polymers using a metallocene catalyst system. An olefinic process stream, containing small amounts of acetylenic impurities, carbon oxides and/or other organic components which are, typically, impurities in cracked gas, passes through at least two zones containing heterogeneous adsorbents. Adsorption is carried out in an essentially dihydrogen-free atmosphere within the initial zone containing a bed of regenerated adsorbent which has retained a substantial amount of carbon monoxide, to effect selective adsorption of the contained acetylenic contaminants with the adsorbent. An effluent mixture which contains less than about 1 part per million by volume of the acetylenic impurities and an amount of carbon monoxide deleterious to a metallocene catalyst system in formation of olefin polymers is contacted in a second zone with an adsorbent capable of effecting selective adsorption of the contained carbon monoxide, thereby obtaining a feedstock which contains less than about 5 parts per million by volume of carbon monoxide and less than about 1 parts per million by volume of the acetylenic impurities. Advantageously, the loaded bed of adsorbent in the first zone is regenerated in the presence of a reducing gas comprising dihydrogen and containing at least 50 parts per million of carbon monoxide, to effect release of the contained acetylenic impurities from the adsorbent.

### FIELD OF THE INVENTION

The field of this invention relates to use of heterogeneous adsorbents in purification of relatively impure olefins, such as are typically produced by thermal cracking of suitable hydrocarbons, to obtain feedstocks suitable for formation of olefin polymers using a metallocene catalyst system. More particularly, this invention concerns purification by passing an olefinic process stream, containing small amounts of acetylenic impurities, carbon oxides and/or other organic components which are, typically, impurities in cracked gas, through at least two zones containing heterogeneous adsorbents. An initial zone contains a bed of regenerated adsorbent which has retained a substantial amount of carbon monoxide to effect selective adsorption of the contained acetylenic contaminants with the adsorbent, and thereby obtain an effluent mixture which contains less than about 1 part per million by volume of the acetylenic impurities and an amount of carbon monoxide deleterious to a metallocene catalyst system in formation of olefin polymers. The effluent mixture is contacted in a second zone with an adsorbent capable of effecting selective adsorption of the contained carbon monoxide, thereby obtaining a feedstock which contains less than about 5 parts per million by volume of carbon monoxide and less than about 1 parts per million by volume of the acetylenic impurities. Advantageously, the loaded bed of adsorbent in the first zone is regenerated in the presence of a reducing gas comprising dihydrogen and containing at least 50 parts per million of carbon monoxide, to effect release of the contained acetylenic impurities from the adsorbent

Processes according to this invention are particularly useful where the olefin being purified is ethylene and/or propylene formed by thermal cracking of hydrocarbon feedstocks.

### BACKGROUND OF THE INVENTION

As is well known, olefins, or alkenes, are a homologous series of hydrocarbon compounds characterized by having a double bond of four shared electrons between two carbon atoms. The simplest member of the series, ethylene, is the largest volume organic chemical produced today. Olefins including, importantly, ethylene, propylene and smaller amounts of butadiene, are converted to a multitude of intermediate and end products on a large scale, mainly polymeric materials.

Commercial production of olefins is, almost exclusively, accomplished by pyrolysis of hydrocarbons in tubular reactor coils installed in externally fired heaters. Thermal cracking feed stocks include streams of ethane, propane or a hydrocarbon liquid ranging in boiling point from light straight-run gasoline through gas oil. Because of the very high temperatures employed, commercial olefin processes invariably coproduce significant amounts of acetylene and methyl acetylene. Required separation of the acetylene from the primary olefin can, considerably, increase the plant cost.

In a typical ethylene plant the cracking section represents about 25 percent of the cost of the unit while the compression, heating, dehydration, recovery and refrigeration sections represent the remaining about 75 percent of the total. This endothermic process is carried out in large pyrolysis furnaces with the expenditure of large quantities of heat which is provided in part by burning the methane produced in the cracking process. After cracking, the reactor effluent is put through a series of separation steps involving cryogenic separation of products such as ethylene and propylene. The total energy requirements for the process are thus very large and ways to reduce it are of substantial commercial interest. In addition, it is of interest to reduce the amount of methane and heavy fuel oils produced in the cracking processor to utilize it other than for its fuel value.

Hydrocarbon cracking is carried out using a feed which is ethane, propane or a hydrocarbon liquid ranging in boiling point from light straight-run gasoline through gas oil. Ethane, propane, liquid naphthas, or mixtures thereof are preferred feed to a hydrocarbon cracking unit. Hydrocarbon cracking is, generally, carried out thermally in the presence of dilution steam in large cracking furnaces which are heated by burning, at least in part, methane and other waste gases from the olefins process resulting in large amounts of NOx pollutants. The hydrocarbon cracking process is very endothermic and requires large quantities of heat per pound of product. However, newer methods of processing hydrocarbons utilizes at least to some extent catalytic processes which are better able to be tuned to produce a particular product slate. The amount of steam used per pound of feed in the thermal process depends to some extent on the feed used and the product slate desired. Typically, steam pressures are in the range of about 30 lbs per sq in to about 80 lbs per sq in, and amounts of steam used are in the range of about 0.2 pounds of steam per pound of feed to 0.7 pounds of per pound of feed. The temperature, pressure and space velocity ranges used in thermal hydrocarbon cracking processes to some extent depend upon the feed used and the product slate desired which are well known as may be appreciated by one skilled in the art.

The type of furnace used in the thermal cracking process is also well known. However the ceramic honeycomb furnace which is described in U.S. Pat. No. 4,926,001, the contents of which patent are specifically incorporated herein by reference, is an example of a new type of cracking which could have a special utility for this process.

Several methods are known for separation of an organic gas containing unsaturated linkages from gaseous mixtures. These include, for instance, cryogenic distillation, liquid adsorption, membrane separation and the so called “pressure swing adsorption” in which adsorption occurs at a higher pressure than the pressure at which the adsorbent is regenerated. Cryogenic distillation and liquid adsorption are common techniques for separation of carbon monoxide and alkenes from gaseous mixtures containing molecules of similar size, e.g., nitrogen or methane. However, both techniques have disadvantages such as high capital cost and high operating expenses. For example, liquid adsorption techniques suffer from solvent loss and need a complex solvent make-up and recovery system.

Molecular sieves which selectively adsorb carbon monoxide from gaseous mixtures by chemisorption are also known. U.S. Pat. No. 4,019,879 and U.S. Pat. No. 4,034,065 refer to use of high silica zeolites, which have relatively high selectivities for carbon monoxide, in the pressure swing adsorption method. However, these zeolites only have moderate capacity for carbon monoxide and more particularly require very low vacuum pressures to recover the adsorbed gases and/or to regenerate the zeolite.

U.S. Pat. No. 4,717,398 describes a pressure swing adsorption process for selective adsorption and subsequent recovery of an organic gas containing unsaturated linkages from gaseous mixtures by passing the mixture over a zeolite ion-exchanged with cuprous ions (Cu I) characterized in that the zeolite has a faujasite type crystalline structure (Y).

Kokai JP Number 50929-1968 describes a method of purifying vinyl compounds containing up to about 10 percent by weight of acetylene compounds including ethyl acetylene, vinyl acetylene and phenyl acetylene whereby the acetylene compounds are adsorbed in an adsorption agent of 1-valent and/or 0-valent copper and/or silver supported on inert carrier such as δ-alumina, silica or active carbon. However, it is well known that acetylene and these acetylene compounds react with copper and/or silver to from copper acetylide or silver acetylide. Both the acetylide of copper and silver are unstable compounds. Because they are explosive under some conditions their possible formation presents safety problems in operation and in handling adsorbent containing such precipitates.

German Disclosure Document 2059794 describes a liquid adsorption process for purification of paraffinic, olefinic and/or aromatic hydrocarbons with an adsorption agent consisting in essence of a complex of a copper (Cu I)-salt with an alkanolamine such as monoethanolamine, monoisopropanolamine, diethanolamine, triethanolamine and arylalkanolmines, and optionally in the presence of a glycol or polyglycol. However, the product stream is contaminated with unacceptable levels of components of the such agents absorbed in the hydrocarbon flow. While such contamination might be removable using an additional bed of silica gel, aluminum oxide or a wide-pored molecular sieve, this would involve additional capital costs, operation expenses and perhaps safety problems.

Processes using heterogeneous adsorbents are known for purification of olefins, such as are typically produced by thermal cracking of suitable hydrocarbon feedstocks, by passing a stream of olefin through a particulate bed of support material on which is dispersed a metallic element. U.S. Pat. No. 6,080,905 and U.S. Pat. No. 6,124,517 in the name of Mark P. Kaminsky, Shiyou Pei, Richard A Wilsak, and Robert E. Whittaker describe adsorption which is carried out in an essentially dihydrogen-free atmosphere within the bed. Adsorption of the contained acetylenic impurities is continued until levels of acetylenic impurities in the effluent stream increase to a predetermined level. Thereafter the resulting bed of adsorbent is regenerated using hydrogen to effect release of the contained acetylenic impurities from the adsorbent. However, there remains a need to increase the capacity of adsorbents for acetylenics whereby the useful life of the adsorbent bed between regenerations is increased.

Olefin-paraffin separations represent a class of most important and also most costly separations in the chemical and petrochemical industry. Cryogenic distillation has been used for over 60 years for these separations. They remain to be the most energy-intensive distillations because of the close relative volatilities. For example, ethane-ethylene separation is carried out at about −25° C. and 320 pounds per square inch gauge pressure (psig) in a column containing over 100 trays, and propane-propylene separation is performed by an equally energy-intensive distillation at about −30° C. and 30 psig.

Impurity refers to compounds that are present in the olefin plant feedstocks and products. Well-defined target levels exist for impurities. Common impurities in ethylene and propylene include: acetylene, methyl acetylene, methane, ethane, propane, propadiene, and carbon dioxide. Listed below are the mole weight and atmospheric boiling points for the light products from thermal cracking and some common compounds potentially found in an olefins unit. Included are some compounds which have similar boiling temperatures to cracked products and may be present in feedstocks or produced in trace amounts during thermal cracking.

| | Mole | Normal Boiling |
|---|---|---|
| Compound | Weight | Point, °C. |
| Hydrogen | 2.016 | −252.8 |
| Nitrogen | 28.013 | −195.8 |
| Carbon monoxide | 28.010 | −191.5 |
| Oxygen | 31.999 | −183.0 |
| Methane | 16.043 | −161.5 |
| Ethylene | 28.054 | −103.8 |
| Ethane | 30.070 | −88.7 |
| Phosphine | 33.970 | −87.4 |
| Acetylene* | 26.038 | −84.0 |
| Carbon dioxide* | 44.010 | −78.5 |
| Radon | 222.00 | −61.8 |
| Hydrogen sulfide | 34.080 | −60.4 |
| Arsine | 77.910 | −55.0 |
| Carbonyl sulfide | 60.070 | −50.3 |
| Propylene | 42.081 | −47.8 |
| Propane | 44.097 | −42.1 |
| Propadiene (PD) | 40.065 | −34.5 |
| Cyclo-propane | 42.081 | −32.8 |
| Methyl acetylene | 40.065 | −23.2 |
| Water | 18.015 | 100. |

*Sublimation temperature

Recently the trend in the hydrocarbon processing industry is to reduce commercially acceptable levels of impurities in major olefin product streams, i.e., ethylene, propylene, and hydrogen. Need for purity improvements is directly related to increasing use of higher activity catalysts for production of polyethylene and polypropylene, and to a limited extent other olefin derivatives.

It is known that acetylenic impurities can be selectively hydrogenated and thereby removed from such product streams by passing the product stream over an acetylene hydrogenation catalyst in the presence of dihydrogen (molecular hydrogen, H₂). However, these hydrogenation processes typically result in the deposition of carbonaceous residues or “green oil” on the catalyst which deactivates the catalyst. Therefore, acetylene hydrogenation processes for treating liquid or liquefiable olefins and diolefins typically include an oxygenation step or a “burn” step to remove the deactivating carbonaceous residues from the catalyst followed by a hydrogen reduction step to reactivate the hydrogenation catalyst. For example, see U.S. Pat. No. 3,755,488 to Johnson et al., U.S. Pat. No. 3,792,981 to Hettick et al., U.S. Pat. No. 3,812,057 to Morgan and U.S. Pat. No. 4,425,255 to Toyoda. However, U.S. Pat. No. 3,912,789 and U.S. Pat. No. 5,332,705 state that by using selected hydrogenation catalysts containing palladium, at least partial regeneration can be accomplished using a hydrogenation step alone at high temperatures (600° F.-700° F.) and in the absence of an oxygenation step.

Selective hydrogenation of the about 2000 to 4000 parts per million of acetylenic impurities to ethylene is, generally, a crucial operation for purification of olefins produced by thermal steam cracking. Typical of a small class of commercially useful catalysts are materials containing very low levels of an active metal supported on an inert carrier, for example a particulate bed having less than about 0.03 percent (300 ppm) palladium supported on the surface skin of carrier pellets having surface area of less than about 10 m²/gm.

Many commercial olefin plants using steam crackers use, generally, front-end acetylene converters, i.e., the hydrogenation unit is fed C₃ and lighter cracked gas which feed has a high enough concentration of hydrogen to easily hydrogenate the acetylenic impurities, however, when run improperly, will also hydrogenate a large fraction of the ethylene and propylene product. Both hydrogenation of acetylene and ethylene are highly exothermic as shown below:

| | |
|---|---|
| C₂ H₂ + H₂ - - - > C₂ H₄ | H = −41 kcal/mole |
| C₂ H₄ + H₂ - - - > C₂ H₆ | H = −32.7 kcal/mole |

Accelerated catalyst deactivation and thermal runaways caused by loss in catalyst selectivity are common problems which plague acetylene converters. Such problems result in unscheduled shutdowns and increased costs to replace deactivated catalyst.

The problem of over-hydrogenation is aggravated because the rate constant for ethylene hydrogenation to ethane is 100 times faster than for the hydrogenation of acetylene to ethylene. As a means to avoid a C₂H₄ hydrogenation thermal runaway, acetylene, carbon monoxide and diolefins concentrations must, therefore, be high enough to cover most of the active sites so that none are left to adsorb ethylene. For example, acetylene, carbon monoxide, methyl acetylene, and propadiene have bond strengths to palladium which are stronger than the ethylene to palladium bonds. Selection of active metal, size of the metal particles and other physical and chemical factors ultimately affect the “operating temperature window” which is the delta of temperature between acetylene conversion to ethylene (typically in a range from about 100° F. to about 150° F.) and thermal runaway where all molecular hydrogen is converted and a large amount of the ethylene is converted to ethane (about 170° F. to about 225° F.). The wider the window, the safer is operation of the unit.

Commercial uses of olefins, particularly, ethylene, propylene and smaller amounts of butadiene, include catalytic conversions of the olefin to a multitude of polymeric products on a large scale. Metallocene catalyst systems represent an increasingly important class of useful catalysts for resin production in industry. Three types of metallocene catalysts are presently used in industry: Kaminsky, combination, and constrained-geometry catalysts.

Metallocene catalyst systems of the Kaminsky type contain two components. One component is a metallocene complex of zirconium, titanium, or hafnium, which usually contains two cyclopentadienyl rings. The other component is either a perfluorinated boron-aromatic compound, an organoaluminum compound, or methylaluminoxane. A large number of metallocene complexes can be used in Kaminsky type catalysts. Their rings contain various alkyl substituents, both linear and cyclic; both rings can be linked together by bridging groups. Among the metallocene compounds, zirconium complexes are preferred on the basis of their exceptionally high activity in ethylene polymerization reactions. While the Kaminsky type catalysts can operate at [A1]:[transition metal] ratios of at least 100, the preferred range is upward from about 1000. The most attractive feature of Kaminsky type is that they are able to produce ethylene copolymers with high compositional uniformity, a particularly important feature for LLDPE resins. However, another distinguishing feature of the Kaminsky type catalysts is high sensitivity of the polymer molecular weight to the presence of dihydrogen, which greatly reduces molecular weights of polymers. and carbon monoxide in the reaction medium.

Combinations of metallocene complexes of zirconium or titanium and perfluorinated aromatic boron compounds are ionic. Theses types of catalysts do not need any organoaluminum compounds; however, monomers of very high purity are required to prevent catalyst poisoning.

The constrained-geometry type of catalysts, contain monocyclopentadienyl derivatives of titanium or zirconium. One of the carbon atoms in the cyclopentadienyl ring is additionally linked to the metal atom by a bridge. The complexes are converted to polymerization catalysts by reacting with methylaluminoxane or by forming ionic complexes with noncoordinative anions. These catalysts have a high capability to copolymerize linear α-olefins with ethylene to produce compositionally uniform ethylene-α olefin copolymers, and can also copolymerize ethylene with styrene and hindered olefins.

It is therefore a general object of the present invention to provide an improved process which overcomes the aforesaid problem of prior art methods, for production of olefins from thermal cracking of hydrocarbon feed stocks which olefin can be used for manufacture of polymeric materials using higher activity catalysts.

More particularly, it is an object of the present invention to provide an improved method for purification of an olefin stream, such as ethylene and/or propylene, containing small amounts of acetylenic impurities, carbon oxides and/or other organic components that are impurities in olefinic process streams.

The selective removal acetylene from polymer-grade olefin by adsorption beneficially replaces an acetylene hydrogenation step, or serves to reduce severity of the acetylene hydrogenation reaction conditions and to produce acetylene-free feedstock suitable for formation of olefin polymers using a metallocene catalyst system. Advantageously, this invention impacts both plant reliability, cost of ethylene production, and product purity.

It is another object of the present invention to provide an improved aforesaid purification method that employs an adsorbent that, even after a substantial period of aging, exhibits ability to withstand repeated regenerations and yet retain useful adsorption capacity.

It is further an object of this invention to provide an improved process for regeneration of adsorbent loaded with acetylenic impurities.

Other objects and advantages of the invention will become apparent upon reading the following detailed description and appended claims.

### SUMMARY OF THE INVENTION

Economical processes are disclosed which obtain purified olefinic product suitable as feedstock for formation of olefin polymer using a metallocene catalyst system by purification of relatively impure olefins such as are produced, for example, by thermal cracking of hydrocarbons. Processes of this invention comprise: (A) providing an impure gaseous mixture comprising at least one olefin of from 2 to about 8 carbon atoms, acetylenic impurities having the same or similar carbon content in an amount of up to about 1 percent by volume based upon the total amount of olefin present and optionally saturated hydrocarbon gases; (B) passing the impure mixture through a first zone containing a bed of regenerated adsorbent which has retained a substantial amount of carbon monoxide, the adsorbent comprising predominantly a support material having high surface area on which is dispersed at least one metallic element in the zero valent state selected from the group consisting of chromium, iron, cobalt, nickel, copper, ruthenium, palladium, silver and platinum, to effect, under conditiors suitable for adsorption within the bed, selective adsorption and/or complexing of the contained acetylenic contaminants with the adsorbent, and thereby obtain an effluent mixture which contains less than about 1 part per million by volume of the acetylenic impurities and an amount of carbon monoxide deleterious to a metallocene catalyst system in formation of olefin polymers; and (C) contacting the effluent mixture in a second zone containing an adsorbent capable of effecting, under conditions suitable for adsorption within the zone, selective adsorption and/or complexing of the contained carbon monoxide with the adsorbent therein, and thereby obtain a feedstock which contains less than about 5 parts per million by volume of carbon monoxide and less than about 1 parts per million by volume of the acetylenic impurities. After levels of the acetylenic impurities in the effluent mixture increase to a limiting level, preferably in a range downward from about 1 parts per million by volume, the resulting bed of adsorbent in the first zone is regenerated in the presence of a reducing gas comprising dihydrogen (molecular hydrogen) and containing at least 50 parts per million of carbon monoxide, to effect release of the contained acetylenic impurities from the adsorbent

Another aspect of special significance is the separation of acetylenic impurities from ethylene or propylene containing small amounts of acetylenic impurities, i.e., less than about 5000 parts per million by weight of one or more acetylenic impurities, and provide, advantageously, purified product containing less than about 1 parts per million by weight, and frequently even less than about 0.5 parts per million by weight.

In another aspect the invention is a process for purification of olefins produced by thermal cracking of hydrocarbons to obtain a feedstock suitable for formation of olefin polymers using a metallocene catalyst system, which purification process comprises: providing an impure gaseous mixture comprising at least about 99 percent by volume of an olefin having from 2 to about 4 carbon atoms, and acetylenic impurities having the same or similar carbon content in an amount in a range upward from about 1 to about 1000 parts per million by volume based upon the total amount of olefin present and optionally saturated hydrocarbon gases; passing the impure mixture through a first zone containing a bed of regenerated adsorbent which has retained a substantial amount of carbon monoxide, the adsorbent comprising predominantly a support material selected from the group alumina, silica, active carbon, clay and zeolites having surface area in a range of from about 10 to about 2,000 square meters per gram as measured by the BET gas adsorption method, on which is dispersed at least one metallic element in the zero valent state selected from the group consisting of chromium, iron, cobalt, nickel, copper, ruthenium, palladium, silver and platinum, to effect, under conditions suitable for adsorption within the bed, selective adsorption and/or complexing of the contained acetylenic contaminants with the adsorbent, and thereby obtain an effluent mixture which contains less than about 1 part per million by volume of the acetylenic impurities and an amount of carbon monoxide in a range upward from about 10 parts per million by volume; and passing the effluent mixture through a second zone containing a bed of adsorbent which comprises at least one metallic element in a positive valent state selected from the group consisting of calcium, titanium, vanadium, chromium, manganese, iron, cobalt, nickel, copper, zinc, zirconium, molybdenum, palladium, silver and tin, to effect, under conditions suitable for adsorption within the zone, selective adsorption and/or complexing of the contained carbon monoxide with the adsorbent therein, and thereby obtain a feedstock which contains less than about 5 parts per million by volume of carbon monoxide and less than about 1 parts per million by volume of the acetylenic impurities. Advantageously, the gaseous mixtures pass though the beds of adsorbent at gas hourly space velocities in a range of from about 0.05 hours⁻¹ to about 20,000 hours⁻¹ measured at standard conditions of 0° C. and 760 mm Hg.

A preferred class of adsorbents useful in the first zone of processes according the invention, comprises at least about 90 weight percent of a gamma alumina having surface area in a range of from about 80 to about 500 square meters per gram as measured by the BET gas adsorption method, and contains less than 500 parts per million by weight of a sulfur-containing component, calculated as elemental sulfur. Advantageously, the metallic element dispersed on the support material in the first zone is at least one element selected from the group consisting of iron, cobalt, nickel, copper, palladium, silver and platinum, and the regenerated absorbent has a dispersed metal content in a range of from about 0.01 to about 10 percent based on the total weight of the adsorbent. More preferred are the adsorbents which comprises at least about 90 weight percent of a gamma alumina having surface area in a range of from about 150 to about 350 square meters per gram as measured by the BET gas adsorption method, and wherein the metal dispersed on the support material is palladium, and the absorbent has a palladium content in a range of from about 0.01 to about 10 percent based on the total weight of the adsorbent.

A preferred class of adsorbents useful in the second zone of processes according the invention, comprises cations of at least one element selected from the group consisting of calcium, titanium, vanadium, chromium, manganese, iron, cobalt, nickel, copper, zinc, zirconium, molybdenum, palladium, silver and tin. Beneficially, the adsorbent in the second zone comprises a solid which has surface area in a range of from about 10 to about 2,000 square meters per gram as measured by the BET gas adsorption method. Advantageously, the adsorbent in the second zone comprises at least one compound selected from the group consisting of calcium carbonate, manganese oxide, oxides of cobalt(II), (III) and (II/III), copper(I) chloride, copper(II) chloride, copper(I) oxide, copper(II) oxide, tin(II) chloride, tin(IV) chloride, palladium chloride, silver nitrate, and zinc oxide. More preferred for use in the second zone are the class of solid adsorbents which comprises cations of copper.

In yet another aspect the invention is a process for purification of a gaseous mixture to obtain a feedstock suitable for formation of polymer using a metallocene catalyst system, which purification process comprises: (a) providing an impure gaseous mixture comprising at least about 99 percent by volume of ethylene, and acetylene impurity in an amount in a range upward from about 1 to about 1000 parts per million by volume based upon the total amount of olefin present and optionally saturated hydrocarbon gases; (b) passing the impure mixture through a first zone containing a bed of adsorbent which has retained a substantial amount of carbon monoxide, the adsorbent comprising at least about 90 weight percent of gamma alumina having surface area in a range of from about 150 to about 350 square meters per gram as measured by the BET gas adsorption method, on which is dispersed palladium in the zero valent state, to effect, under conditions suitable for adsorption within the bed, selective adsorption and/or complexing of the contained acetylenic contaminants with the adsorbent, and thereby obtain an effluent mixture which contains less than about 1 part per million by volume of the acetylenic impurities and an amount of carbon monoxide in a range upward from about 10 parts per million by volume; (c) passing the effluent mixture through a second zone containing a bed of adsorbent which comprises at least one metallic element in a positive valent state selected from the group consisting of calcium, titanium, vanadium, chromium, manganese, iron, cobalt, nickel, copper, zinc, zirconium, molybdenum, palladium, silver and tin, to effect, under conditions suitable for adsorption within the zone, selective adsorption and/or complexing of the contained carbon monoxide with the adsorbent therein, and thereby obtain a feedstock which contains less than about 5 parts per million by volume of carbon monoxide and less than about 1 parts per million by volume of the acetylenic impurities; (d) effecting, in the presence of an essentially dihydrogen-free atmosphere within the bed in the first zone, selective adsorption and/or complexing of the contained acetylenic impurities with the adsorbent, until levels of the acetylenic impurities in the effluent mixture increase to a limiting level in a range downward from about 1 parts per million by volume; and thereafter (e) regenerating the resulting bed of adsorbent in the first zone in the presence of a reducing gas comprising dihydrogen and containing at least 50 parts per million of carbon monoxide, to effect release of the contained acetylenic impurities from the adsorbent.

For a more complete understanding of the present invention, reference should now be made to the embodiments illustrated in greater detail and described below by way of examples of the invention.

### BRIEF DESCRIPTION OF THE INVENTION

Processes of this invention are particularly suitable for use in purification of aliphatically unsaturated organic compounds produced, generally, by thermal cracking of hydrocarbons.

Aliphatically unsaturated compounds of most interest with regard to purification by the method of the present invention, have two to about eight carbon atoms, preferably two to about four carbon atoms, and more preferably ethylene or propylene. The separation of acetylenic impurities from ethylene or propylene which may be contained in admixtures with other normally gaseous materials, such as one or more of ethane, methane, propane and oxides of carbon is of particular importance. For example mixtures serving as a source of ethylene containing feed for the process may contain about 1 to about 99 weight percent ethylene, about 0 to about 50 weight percent ethane and/or about 0 to about 50 weight percent methane.

Generally acetylenic impurities described in this invention are expressed by the formula

R—C≡CH

where R is hydrogen or a hydrocarbon group of up to 6 carbon atoms.

It is desirable to treat the gaseous mixture used in the process of the present invention to remove any gaseous hydrogen. The amount of dihydrogen in the gaseous mixture should suitably be reduced to below 10 parts per million by weight, preferably below 2 parts per million by weight and most preferably below 1 parts per million by weight, prior to contact with the adsorbent.

Any mercury-containing, arsenic-containing, and sulfur-containing components, e.g., hydrogen sulfide, present in the gaseous mixture fed to the particulate bed of adsorbent should suitably be removed therefrom in any known manner in order to avoid the risk of poisoning the dispersed metal. The hydrocarbon mixture used in the process of the present invention is suitably a cracked gas from which the majority of the C₅ and higher hydrocarbons have been removed. The gaseous mixture may thus comprise ethylene, propylene, butenes, methane, ethane, propane and butane.

In preferred embodiments of processes according to the invention, the olefin in the gaseous mixture being purified is predominantly ethylene or propylene, the gaseous mixture contains less than about 0.5 parts per million by volume of hydrogen and less than about 1 parts per million by volume of mercury-containing, arsenic-containing, and sulfur-containing components, each calculated as the element, and wherein the gaseous mixture, while passing through the bed, is at temperatures in a range upward from about 20° C. to about 100° C., preferably in a range of from about 25° C. to about 65° C.

The gaseous mixture used in the process of the present invention may also comprise water and may optionally be saturated with water.

According to the invention, the contacting in a second zone of the effluent mixture from a first zone, is with any adsorbent capable of effecting, under conditions suitable for adsorption within the zone, selective adsorption and/or complexing of the contained carbon monoxide with the adsorbent therein. Adsorbents suitable the for selective adsorption and/or complexing of carbon monoxide according to the invention comprise cations, e.g. elements in a positive valent state. Advantageously these adsorbents include one or more cations selected from the group consisting of elements having atomic numbers from 20 to 50. More particularly, a suitable adsorbent comprises at least one element selected from the class of elements having atomic numbers 20, 22 to 30 inclusive, 40, 42, 46, 47 and 50 in a positive valent state. Preferred adsorbents include metal salts of titanium, vanadium, chromium, manganese, iron, cobalt, nickel, copper, zinc, zirconium, molybdenum, palladium, silver and tin, activated carbon, and Faujasite zeolite. Other preferred adsorbents comprise an oxide of manganese, cobalt, copper, and zinc, a chloride of copper, tin, and palladium, nickel ferrite, calcium carbonate, silver nitrate. Complexes of manganese, iron and/or tin, copper/ammonium salt solutions, and copper(I) organometallic complexes, such as cuprous tetrachloroaluminate toluene complex (CuAlCl₄ C₆H₅CH₃ in a toluene solvent), can likewise be used in accordance with the invention.

Adsorbents for selective adsorption and/or complexing of carbon monoxide are used according to the invention in any active form suitable for contacting with a gaseous stream, typically in a non-gaseous phase. While liquids containing an active component may be useful, adsorbents are preferably used in solid form.

Broadly, according to the present invention, there is provided a particulate bed of adsorbent comprising predominantly a support material having high surface area on which is dispersed at least one metallic element selected from the group consisting of chromium, iron, cobalt, nickel, copper, ruthenium, palladium, silver, and platinum. Suitable adsorbents exhibit, in the presence of an essentially dihydrogen-free atmosphere within the bed, selective adsorption and/or complexing of the acetylenic impurities with the adsorbent. According to the present invention dispersed metal content is in a range of from about 0.01 to about 40 percent based on the total weight of the adsorbent. Preferably dispersed metal content is in a range of from about 0.01 to about 20 percent based on the total weight of the adsorbent.

The adsorbent can, optionally, further comprise one or more elements selected from the group consisting of lithium, sodium, potassium, zinc, molybdenum, tin, tungsten, and iridium, dispersed on the support material. Preferably the adsorbent further comprises a member selected from the group consisting of lithium, sodium, potassium, zinc, molybdenum, and tin dispersed on the support material.

For processes according to invention the metal dispersed on the support material is, advantageously, at least one element selected from the group consisting of chromium, iron, cobalt, nickel, copper, ruthenium, palladium, silver and platinum, and the absorbent has a dispersed metal content in a range of from about 0.05 to about 20 percent based on the total weight of the adsorbent.

Another class adsorbents useful for processes according to invention comprises a dispersion of copper or silver and one metallic element selected from the group consisting of chromium, iron, cobalt, nickel, ruthenium, palladium, and platinum, preferably palladium.

More preferred for processes according to this invention are adsorbents having palladium metal dispersed on the support, and the absorbent has a palladium content in a range of from about 0.05 to about 10 percent, more preferred palladium content in a range of from about 0.1 to about 5.0 percent based on the total weight of the adsorbent.

High metal dispersion and loading resulted in higher metal surface area. Capacity of an adsorbent is, typically, related directly to metal surface area. Any method which increases and/or maintains high metal surface area is, therefore, beneficial to achieving high acetylene adsorption capacity.

Preferred for processes according to this invention are adsorbents having a dispersion value of at least about 10 percent, preferably in a range upward from about 20 percent to about 80 percent. Dispersion is a measure of the accessibility of the active metals on the adsorbent. Such dispersion methods are discussed in H. C. Gruber's, Analytical Chemistry, Vol. 13, p. 1828, (1962). The absorbents for use in this invention were analyzed for dispersion using a pulsed carbon monoxide technique as described in more detail in the Examples. Palladium containing adsorbents having large dispersion values are desired because more of the palladium metal is available for reaction.

Support materials are, advantageously, selected from the group consisting of alumina, silica, carbon, clay and zeolites (molecular sieves). Surface areas of support materials are, preferably, in a range of from about 10 to about 2,000 square meters per gram as measured by the BET gas adsorption method.

A preferred class of active carbons useful herein are materials disclosed in commonly assigned U.S. Pat. No. 4,082,694 to Arnold N. Wennerberg and Thomas M. O'Grady, which patent is incorporated herein by reference. Such suitable active carbon products are produced from carbonaceous material by a staged temperature process which provides improved yield and processability during manufacture. A source of carbonaceous material, such as crushed coal, coal coke, petroleum coke or a mixture thereof, is heated with agitation in the presence of a substantial weight ratio of potassium hydroxide at a first lower temperature to dehydrate the combination. Thereafter the temperature is raised to a second higher temperature to activate the combination which is thereafter cooled and washed to remove inorganic matter and form a high surface area active carbon having a cage-like structure exhibiting micro-porosity, good bulk density and Total Organic Carbon Index.

Active carbon products for use as supports according to this invention have, preferably, an effective surface area greater than about 2,300 square meters per gram and, more preferably, greater than about 2,700 square meters per gram and, most preferably, above about 3,000 square meters per gram as measured by the BET method. Active carbon products for use as supports have, typically, a bulk density greater than about twenty-five hundredths grams per cubic centimeter and, preferably greater than about twenty-seven hundredths grams per cubic centimeter and, more preferably, above about three-tenths gram per cubic centimeter. Further, useful active carbon products preferably have a Total Organic Carbon Index greater than about 300, more preferably, greater than about 500 and, most preferably, greater than about 700.

Generally, the term “molecular sieve” includes a wide variety of positive-ion-containing crystalline materials of both natural and synthetic varieties. They are generally characterized as crystalline aluminosilicates, although other crystalline materials are included in the broad definition. The crystalline aluminosilicates are made up of networks of tetrahedra of SiO₄ and AlO₄ moieties in which the silicon and aluminum atoms are cross-linked by the sharing of oxygen atoms. The electrovalence of the aluminum atom is balanced by the use of positive ions, for example, alkali-metal or alkaline-earth-metal cations.

Zeolitic materials, both natural and synthetic, useful herein have been demonstrated in the past to have catalytic capabilities for many hydrocarbon processes. Zeolitic materials, often referred to as molecular sieves, are ordered porous crystalline aluminosilicates having a definite structure with large and small cavities interconnected by channels. The cavities and channels throughout the crystalline material are generally uniform in size allowing selective separation of hydrocarbons. Consequently, these materials in many instances have come to be classified in the art as molecular sieves and are utilized, in addition to the selective adsorptive processes, for certain catalytic properties. The catalytic properties of these materials are also affected, to some extent, by the size of the molecules which are allowed selectively to penetrate the crystal structure, presumably to be contacted with active catalytic sites within the ordered structure of these materials.

In the past various molecular sieve compositions natural and synthetic have been found to be useful for a number of hydrocarbon conversion reactions. Among these are alkylation, aromatization, dehydrogenation and isomerization. Among the sieves which have been used are Type A, X, Y and those of the MFI crystal structure, as shown in “Atlas of Zeolite Structure Types,” Second Revised Edition 1987, published on behalf of the Structure Commission of the International Zeolite Associates and incorporated by reference herein. Representative of the last group are ZSM-5 and AMS borosilicate molecular sieves.

Prior art developments have resulted in the formation of many synthetic crystalline materials. Crystalline aluminosilicates are the most prevalent and, as described in the patent literature and in the published journals, are designated by letters or other convenient symbols. Exemplary of these materials are Zeolite A (Milton, in U.S. Pat. No. 2,882,243), Zeolite X (Milton, in U.S. Pat. No. 2,882,244), Zeolite Y (Breck, in U.S. Pat. No. 3,130,007), Zeolite ZSM-5 (Argauer, et al., in U.S. Pat. No. 3,702,886), Zeolite ZSM-11 (Chu, in U.S. Pat. No. 3,709,979), Zeolite ZSM-12 (Rosinski, et al., in U.S. Pat. No. 3,832,449), and others.

Manufacture of the ZSM materials utilizes a mixed base system in which sodium aluminate and a silicon containing material are mixed together with sodium hydroxide and an organic base, such as tetrapropylammonium hydroxide and tetrapropylammonium bromide, under specified reaction conditions, to form the crystalline aluminosilicate, preferably a crystalline metallosilicate exhibiting the MFI crystal structure.

A preferred class of molecular sieves useful, according to the present invention, are crystalline borosilicate molecular sieves disclosed in commonly assigned U.S. Pat. No. 4,268,420, U.S. Pat. No. 4,269,813, U.S. Pat. No. 4,292,457, and U.S. Pat. No. 4,292,458 to Marvin R. Klotz, which are incorporated herein by reference.

### BRIEF DESCRIPTION OF PREFERRED EMBODIMENTS OF THE INVENTION

While this invention is susceptible of embodiment in many different forms, this specification discloses only some specific forms as an example of the use of the invention. In particular, preferred embodiments of the invention for purification of a gaseous mixture comprising olefin preferably an olefin of from two to about eight carbon atoms having a single double bond, acetylenic impurities having the same or similar carbon content and optionally alkanes (paraffin hydrocarbons) and/or alkenes having more than one double bond (di- or tri-olefin hydrocarbons) produced by thermal cracking of hydrocarbons are illustrated and described. The invention is not intended to be limited to the embodiments so described, and the scope of the invention will be pointed out in the appended claims. The apparatus of this invention is used with certain conventional components the details of which, although not fully illustrated or described, will be apparent to those having skill in the art and an understanding of the necessary function of such components.

More specifically an integrated olefin purification system including: one or more optional heat exchangers for controlling temperature of the gaseous feedstream to temperatures in a range from about 20° C. to about 100° C., adsorption vessels containing particulate beds of a suitable solid adsorbents, and means for analysis of feed and effluent streams, such as an on-line analytical system.

During operation of the integrated olefin purification system, a gaseous mixture containing less than about 500 parts per million by weight of the acetylene and carbon monoxide impurities formed by chemical conversions in commercial thermal cracking processes, is, for example ethylene fed from the overhead of a C2 distillation tower or intermediate storage through a feed exchanger to control temperature during adsorption. Effluent from the feed exchanger flows through the first of two adsorption vessels which contain beds of a suitable solid adsorbents.

During operation the gaseous mixture passes though the beds of particulate adsorbent at gas hourly space velocities in a range of from about 0.05 hours⁻¹ to about 20,000 hours⁻¹ and even higher, preferably from about 0.5 hours⁻¹ to about 10,000 hours⁻¹.

Compositions of the gaseous feed and effluent of each adsorption vessel is monitored by on-line analytical system. While levels of acetylenic impurities in the effluent from the first adsorption vessel in purification service are in a range downward from a predetermined level, the effluent, polymer grade ethylene, flows through the second vessel and directly to formation of olefin polymers with a metallocene catalyst system, or to storage. When the level of acetylenic impurities in the effluent of a first adsorption vessel in purification service reaches or exceeds the predetermined level, that adsorption vessel is isolated from the process flow, and thereafter the resulting bed of loaded adsorbent is treated to effect release of the contained acetylenic impurities from the adsorbent by hydrogenation. Advantageously the resulting bed of adsorbent in the first vessel is thereafter regenerated in the presence of a reducing gas comprising dihydrogen and containing at least 50 parts per million of carbon monoxide, to effect release of the contained acetylenic impurities from the adsorbent

Suitable absorbents for used in the first zone have capacity to treat from about 300 to about 40,000 pounds of olefin feed per pound of adsorbent where the olefin feed contains about 0.5 parts per million (ppm) acetylene. Approximately 5×10⁻⁴ pounds of acetylene to about 1×10⁻² pounds are, advantageously, adsorbed per pound of adsorbent before regeneration is required.

During continuous operation of this embodiment, the time required for treating, alternately, of the loaded adsorbent to effect release of the contained acetylenic impurities from the adsorbent by hydrogenation, is provided by using two or more independent adsorption vessels containing beds. Regenerations are, advantageously, performed according to this invention in three steps.

During the first stage of regeneration dry inert gas, such as methane, ethane, or nitrogen which is, preferably, free of carbon oxides, unsaturated hydrocarbons and hydrogen is fed, from, for example a nitrogen gas supply system exchanger to control temperature during regeneration. The dry inert gas flows through the bed of loaded adsorbent thereby purging gaseous hydrocarbons therefrom to disposal.

During the second stage of regeneration a reducing gas stream comprising dihydrogen and containing at least 50 parts per million of carbon monoxide, to effect release of the contained acetylenic impurities from the adsorbent. Preferably the reducing gas stream comprising predominately dihydrogen containing from about 50 to 500 parts per million of carbon monoxide.

Where heating of the regeneration gas is desired, rates of temperature increase during the second stage of regeneration are, preferably, controlled to rates of less than about 11° C. per minute (about 20° F. per minute) while increasing temperature in the range of from about 4° C. to about 200° C. (about 40° F. to about 400° F.). Pressures of the hydrogen-rich reducing gas during the second stage of regeneration are, advantageously, in a range from about 5 psig to about 500 psig. While the reducing gas is flowing through the adsorbent bed, effluent gas composition is, periodically, monitored with gas analyzer. Second stage of regeneration is complete when C2+ hydrocarbon levels in the effluent gas from the bed have been reduced to C2+ hydrocarbon levels in the feed.

Third stage regeneration involves purging all gaseous hydrogen from the adsorption vessel with an inert gas, e.g. nitrogen with or without a saturated hydrocarbon gas such as methane or ethane, while the vessel is at temperatures in a range upward from about 60° C. (140° F.). During this third stage of regeneration flow of inert gas, at or below ambient temperature and about 5 to about 100 psig, cools the vessel to about ambient temperature thereby completing the regeneration process.

Surface area of adsorbents can be determined by the Brunaur-Emmett-Teller (BET) method or estimated by a simpler Point B method. Adsorption data for nitrogen at the liquid nitrogen temperature, 77 K, are usually used in both methods. The Brunaur-Emmett-Teller equation, which is well known in the art, is used to calculate the amount of nitrogen for mono-layer coverage. The surface area is taken as the area for mono-layer coverage based on the nitrogen molecular area, 16.2 square Angstroms, obtained by assuming liquid density and hexagonal close packing. In the Point B method, the initial point of the straight portion of the Type II isotherm is taken as the completion point for the mono-layer. The corresponding amount adsorbed multiplied by molecular area yields the surface area.

Dispersion and surface area of active metal sites was determined by carbon monoxide chemisorption using a Pulse Chemisorb 2700 (Micromeritics). In this procedure, approximately 4 gram samples were purged with helium carrier gas, calcined in air at 500° C. for 1 hr, purged with helium, reduced in hydrogen at 500° C., purged with helium, and cooled to room temperature. The sample was treated with 49.5 percent carbon monoxide in helium and the dosed with 0.045 mL pulses of 49.5 percent carbon monoxide (CO), balance nitrogen, and the carbon monoxide uptake was measured by a thermal conductivity cell. Palladium dispersion values were calculated assuming one carbon monoxide molecule per palladium atom. Palladium loadings are weight percent palladium metal.

In characterizing the pore volume, both total pore volume and its distribution over the pore diameter are needed. The total pore volume is usually determined by helium and mercury densities or displacements. Helium, because of its small atomic size and negligible adsorption, gives the total voids, whereas mercury does not penetrate into the pores at ambient pressure and gives inter-particle voids. The total pore volume equals the difference between the two voids.

Palladium on a high-surface-area γ-Al₂O₃ is a preferred adsorbent for purification of olefins in accordance with this invention. In order to introduce palladium and/or other suitable metal ions on a high-surface-area γ-Al₂O₃, any known technique for monolayer dispersion can be employed. The phenomenon of spontaneous dispersion of metal oxides and salts in monolayer or submonolayer forms onto surfaces of inorganic supports with high surface areas has been studied extensively in the literature (e.g., Xie and Tang, 1990).

### EXAMPLES OF THE INVENTION

The following Examples will serve to illustrate certain specific embodiments of the herein disclosed invention. These Examples should not, however, be construed as limiting the scope of the novel invention as there are many variations which may be made thereon without departing from the spirit of the disclosed invention, as those of skill in the art will recognize.

### Example 1

This example illustrates the procedure used to initially reduce a commercially available adsorbent, and then demonstrates use of an adsorption bed operating at 49° C. after the bed was regenerated with a reducing gas comprising dihydrogen and essentially free of carbon monoxide. It is noted that no carbon monoxide was present in the effluent gas from this adsorption bed.

Initial Reduction of Pd/γ-Al₂O₃ Adsorbent.

A 50 mL TEFLON-lined stainless steel pressure vessel was loaded with 18.88 gm of commercially available adsorbent (about 41 mL of 0.36 percent palladium on γ-Al₂O₃), and a centrally disposed thermocouple system to monitor bed temperatures. After this adsorption vessel was connected into a gas adsorption unit which provided required control of feed gases, temperatures, pressures and analytical means, the adsorbent bed was run in the down-flow mode. Nitrogen was purged through the vessel before reducing the oxidized PdO/γ-Al₂O₃ adsorbent by heating to 42° C. in a flow of hydrogen. A circulating water bath was used to supply heated needed during reduction at 80 psig with hydrogen flowrates of around 250 mL/min. After 16 hours hydrogen flow was replaced with nitrogen flow. The vessel was maintained at a temperature of about 49° C. during the subsequent adsorption process.

First Period of Adsorption for Pd/γ-Al₂O₃ Adsorbent.

After analysis of the effluent gases showed that hydrogen had been purged from the vessel, a feed mixture which contained 208 ppm acetylene in balance of ethylene was introduced into the vessel and the vessel was pressurized to 200 psig. During adsorption the flow rate of the acetylene/ethylene mixture was 201 mL/min and operating conditions of pressure and temperature were controlled at 200 psig and 49° C. By periodical analysis of effluent gas using an on-line gas chromatograph, acetylene was detected (less than about 0.5 ppm acetylene) breaking through the bed of adsorbent after a total of about 103 L (1 atm and 21° C.) of feed gas was treated. In this first period of adsorption, the adsorbent exhibited a capacity of about 0.31 mL of acetylene per mL of adsorbent.

Initial Regeneration of Pd/γ-Al₂O₃ Adsorbent.

After flow of the acetylene/hydrogen/ethylene mixture was stopped, the vessel was depressured to 1 atm and nitrogen was purged through the vessel for about 15 minutes. The vessel was maintained at 49° C. using the circulating water bath. The adsorbent was regenerated using with a reducing gas comprising dihydrogen and essentially free of carbon monoxide at a flow rate of 250 mL/min at 80 psig for about 16 hours. After regeneration for this period, hydrogen flow was replaced with nitrogen flow.

Second Period of Adsorption for Pd/γ-Al₂O₃ Adsorbent.

After analysis of the effluent gases showed that hydrogen had been purged from the vessel, a feed mixture which contained 208 ppm acetylene in balance ethylene was introduced into the vessel and the vessel was pressurized to 200 psig. During adsorption the flow rate of the acetylene/ethylene mixture was 202 mL/min and operating conditions of pressure and temperature were controlled at 200 psig and 49° C. Periodical analysis of the effluent gas was made using an on-line gas chromatograph. No acetylene was detected in the effluent from the adsorbent bed until a total of 96 L (1 atm and 21° C.) of feed gas had been treated. No CO was detected in the effluent from the adsorbent bed at any time. In this example the adsorbent exhibited a capacity of about 0.23 mL of acetylene per mL of adsorbent.

### Example 2

This example demonstrates use of an adsorption bed operating at 49° C. after the bed was regenerated with a reducing gas comprising dihydrogen and about 300 ppm by volume of carbon monoxide. It clearly shows that when carbon monoxide is present in the regeneration gas, carbon monoxide is also detected in the effluent gas from the adsorption bed during the subsequent adsorption cycle.

Flow of the acetylene/ethylene mixture to the spent adsorbent bed from second period of adsorption was stopped, the vessel was depressured to 1 atm and nitrogen was purged through the vessel for about 15 minutes. The vessel was maintained at 49° C. using the circulating water bath. The adsorbent was regenerated using a reducing gas containing 21.1 mol percent dihydrogen, 282 ppm by volume of carbon monoxide, 4.77 mol percent ethylene (C₂H₄), in balance methane at a flow rate of 250 mL/min at 80 psig for about 17 hours. After regeneration for this period, regeneration gas flow was replaced with nitrogen flow.

After analysis of the effluent gases showed that the regeneration gas had been purged from the vessel, a feed mixture which contained 180 ppm acetylene in balance ethylene was introduced into the vessel and the vessel was pressurized to 200 psig. During adsorption the flow rate of the acetylene/ethylene mixture was 202 mL/min and operating conditions of pressure and temperature were controlled at 200 psig and 49° C. Periodical analysis of the effluent gas was made using an on-line gas chromatograph. No acetylene was detected in the effluent from the adsorbent bed until a total of 159 L (1 atm and 21° C.) of feed gas had been treated. In this example of the invention, the adsorbent exhibited a capacity of about 0.38 mL of acetylene per mL of adsorbent. The initial gas chromatograph of the adsorbent bed effluent showed 134 ppm of carbon monoxide. All subsequent analyses showed between 15 to 30 ppm of carbon monoxide in the effluent of the adsorption bed.

### Comparative Examples

A. Preparation of Silver/Zeolite Adsorbent and Regeneration in Air

A sample of silver-exchanged zeolite (Crossfield CG-Z/Ag-102) was prepared according to the following method:

i. 100 mL of 0.3N silver nitrate (AgNO₃) solution was placed in a bottle;

ii. The temperature of the AgNO₃ solution was raised to 70° C.;

iii. While stirring the hot solution, 10 g of zeolite X was added to the hot solution;

iv. Temperature and stirring were maintained for two hours; and

v. Thereafter, the sample was filtered, washed and dried.

This sample of silver-exchanged zeolite was pre-saturated with 3000 ppm acetylene at 25° C. and 115 psig. An ex-situ regeneration in air was carried out at 200° C. and adsorption capacity was determined at 25° C. and 115 psig using 60 ppm acetylene in balance of ethylene. Only 25 percent of the original adsorption capacity was restored. Furthermore, the adsorption efficiency dropped from 100 percent to between 90 and 95 percent of the initial level. Other attempts to regenerate the material by varying regeneration time and temperature, saturation gas composition, and regeneration gas composition were even less successful.

By way of contrast, our invention has been demonstrated to be completely regenerable under commercial conditions, and to lose none of its adsorption efficiency after regeneration.

B. Polymerization of Ethylene Containing less than 0.4 ppm of Acetylene

This example demonstrates a standard gas phase polymerization in a 1 liter autoclave reactor equipped with a paddle stirrer, an external water jacket for temperature control, a septum inlet, and regulated supplies of dry nitrogen, ethylene and hydrogen. The reactor contained 40 g of ground polystyrene to aid stirring in the gas phase. The reactor and its contents were thoroughly dried and degassed at 85° C. by the addition of 2 cc of an 0.5 M hexane solution of triethyl aluminum. The contents were stirred vigorously for several minutes.

A standard Ziegler-Natta catalyst was injected into the reactor and the reactor was pressured to 200 psig with ethylene containing less than 0.4 ppm acetylene. The polymerization was continued for about 10 minutes while maintaining the temperature and pressure. The reaction was stopped with venting and cooling to yield 9 g of typically white polyethylene

C. Polymerization of Ethylene Containing 100 ppm of Acetylene

Another polymerization was performed as in Comparative Example B except that the ethylene contained 105 ppm of acetylene. The 8.5 g of polymer recovered had a distinct, unacceptable, blue color.

D. Polymerization of Ethylene with a Metallocene Catalyst System

This example demonstrates another standard gas phase polymerization in a 1 liter autoclave reactor equipped with a paddle stirrer, an external water jacket for temperature control, a septum inlet, and a regulated supplies of dry nitrogen, ethylene and hydrogen. The reactor contained 40 g of ground polystyrene to aid stirring in the gas phase. The reactor and its contents were thoroughly dried and degassed at 85° C. by the addition of 2 cc of an 0.5 M hexane solution of methyl alumoxane and trimethyl aluminum. The contents were stirred vigorously for several minutes.

A standard supported zirconium metallocene catalyst was injected into the reactor and the reactor was pressured to 200 psig with pure ethylene. The polymerization was continued for about 10 minutes while maintaining the temperature and pressure. The reaction was stopped with venting and cooling to yield 12 g of typically white polyethylene with a molecular weight of 146,000.

E. Polymerization of Ethylene Containing 100 ppm of Acetylene

Polymerization was performed as in the example above except that the ethylene contained 105 ppm acetylene. The yield decrease to 4.8 g of polyethylene which had an unacceptable low molecular weight of 85,000.

Examples have been presented and hypotheses advanced herein in order to better communicate certain facets of the invention. The scope of the invention is determined solely by the scope of the appended claims.

For the purposes of the present invention, “predominantly” is defined as more than about fifty per cent. “Substantially” is defined as occurring with sufficient frequency or being present in such proportions as to measurably affect macroscopic properties of an associated compound or system. Where the frequency or proportion for such impact is not clear substantially is to be regarded as about twenty per cent or more. The term “Essentially” is defined as absolutely except that small variations which have no more than a negligible effect on macroscopic qualities and final outcome are permitted, typically up to about one percent.

That which is claimed is:

1. A process for purification of an olefin stream to obtain a feedstock suitable for formation of olefin polymers using a metallocene catalyst system, which purification process comprises:
providing an impure gaseous mixture comprising at least one olefin of from 2 to about 8 carbon atoms, acetylenic impurities having the same or similar carbon content in an amount of up to about 1 percent by volume based upon the total amount of olefin present and optionally saturated hydrocarbon gases;
passing the impure mixture through a first zone containing a bed of regenerated adsorbent which has retained a substantial amount of carbon monoxide, the adsorbent comprising predominantly a support material having high surface area on which is dispersed at least one metallic element in the zero valent state selected from the group consisting of chromium, iron, cobalt, nickel, copper, ruthenium, palladium, silver and platinum, to effect, under conditions suitable for adsorption within the bed, selective adsorption and/or complexing of the contained acetylenic contaminants with the adsorbent, and thereby obtain an effluent mixture which contains less than about 1 part per million by volume of the acetylenic impurities and an amount of carbon monoxide deleterious to a metallocene catalyst system in formation of olefin polymers; and
contacting the effluent mixture in a second zone containing an adsorbent capable of effecting, under conditions suitable for adsorption within the zone, selective adsorption and/or complexing of the contained carbon monoxide with the adsorbent therein, and thereby obtain a feedstock which contains less than about 5 parts per million by volume of carbon monoxide and less than about 1 parts per million by volume of the acetylenic impurities.

2. The process according to claim 1 wherein the adsorbent in the second zone comprises cations of at least one element selected from the group consisting of calcium, titanium, vanadium, chromium, manganese, iron, cobalt, nickel, copper, zinc, zirconium, molybdenum, palladium, silver and tin.

3. The process according to claim 1 wherein the adsorbent in the second zone comprises a solid which has surface area in a range of from about 10 to about 2,000 square meters per gram as measured by the BET gas adsorption method.

4. The process according to claim 3 wherein the metallic element dispersed on the support material in the first zone is at least one element selected from the group consisting of iron, cobalt, nickel, copper, palladium, silver and platinum, and the regenerated absorbent has a dispersed metal content in a range of from about 0.01 to about 10 percent based on the total weight of the adsorbent.

5. The process according to claim 4 wherein the gaseous mixtures pass though the beds of adsorbent at gas hourly space velocities in a range of from about 0.05 hours⁻¹ to about 20,000 hours⁻¹ measured at standard conditions of 0° C. and 760 mm Hg.

6. The process according to claim 1 wherein the adsorbent in the second zone comprises at least one compound selected from the group consisting of calcium carbonate, manganese oxide, oxides of cobalt(II), (III) and (II/III), copper(I) chloride, copper(II) chloride, copper(I) oxide, copper(II) oxide, tin(II) chloride, tin(IV) chloride, palladium chloride, silver nitrate, and zinc oxide.

7. The process according to claim 6 regenerated adsorbent in the first zone comprises at least about 90 weight percent of a gamma alumina having surface area in a range of from about 80 to about 500 square meters per gram as measured by the BET gas adsorption method, the metallic element dispersed on the alumina is palladium, and the absorbent has a palladium content in a range of from about 0.01 to about 10 percent based on the total weight of the adsorbent.

8. The process according to claim 1 wherein the regenerated adsorbent in the first zone comprises at least about 90 weight percent of a gamma alumina having surface area in a range of from about 150 to about 350 square meters per gram as measured by the BET gas adsorption method, the metallic element dispersed on the support material is palladium, and the absorbent has a palladium content in a range of from about 0.01 to about 10 percent based on the total weight of the adsorbent.

9. The process according to claim 8 wherein the adsorbent in the second zone is a solid and comprises cations of copper.

10. A process for purification of olefins produced by thermal cracking of hydrocarbons to obtain a feedstock suitable for formation of olefin polymers using a metallocene catalyst system, which purification process comprises:
providing an impure gaseous mixture comprising at least about 99 percent by volume of an olefin having from 2 to about 4 carbon atoms, and acetylenic impurities having the same or similar carbon content in an amount in a range upward from about 1 to about 1000 parts per million by volume based upon the total amount of olefin present and optionally saturated hydrocarbon gases;
passing the impure mixture through a first zone containing a bed of regenerated adsorbent which has retained a substantial amount of carbon monoxide, the adsorbent comprising predominantly a support material selected from the group alumina, silica, active carbon, clay and zeolites having surface area in a range of from about 10 to about 2,000 square meters per gram as measured by the BET gas adsorption method, on which is dispersed at least one metallic element in the zero valent state selected from the group consisting of chromium, iron, cobalt, nickel, copper, ruthenium, palladium, silver and platinum, to effect, under conditions suitable for adsorption within the bed, selective adsorption and/or complexing of the contained acetylenic contaminants with the adsorbent, and thereby obtain an effluent mixture which contains less than about 1 part per million by volume of the acetylenic impurities and an amount of carbon monoxide in a range upward from about 10 parts per million by volume; and
passing the effluent mixture through a second zone containing a bed of adsorbent which comprises at least one metallic element in a positive valent state selected from the group consisting of calcium, titanium, vanadium, chromium, manganese, iron, cobalt, nickel, copper, zinc, zirconium, molybdenum, palladium, silver and tin, to effect, under conditions suitable for adsorption within the zone, selective adsorption and/or complexing of the contained carbon monoxide with the adsorbent therein, and thereby obtain a feedstock which contains less than about 5 parts per million by volume of carbon monoxide and less than about 1 parts per million by volume of the acetylenic impurities.

11. The process according to claim 10 wherein the adsorbent in the second zone comprises an oxide of at least one element selected from the group consisting of manganese, cobalt, copper, and zinc.

12. The process according to claim 11 wherein the gaseous mixture passes though the beds of adsorbent at space velocities in a range of from about 0.05 hours⁻¹ to about 20,000 hours⁻¹ measured at standard conditions of 0° C. and 760 mm Hg

13. The process according to claim 11 wherein the adsorbent in the first zone comprises at least about 90 weight percent of a gamma alumina having surface area in a range of from about 80 to about 500 square meters per gram as measured by the BET gas adsorption method, and contains less than 500 parts per million by weight of a sulfur-containing component, calculated as elemental sulfur.

14. process according to claim 10 wherein the adsorbent in the second zone comprises at least one compound selected from the group consisting of calcium carbonate, manganese oxide, oxides of cobalt(II), (III) and (II/III), copper(I) chloride, copper(II) chloride, copper(I) oxide, copper(II) oxide, tin(II) chloride, tin(IV) chloride, palladium chloride, silver nitrate, and zinc oxide.

15. The process according to claim 10 wherein the adsorbent in the second zone comprises cations of copper.

16. The process according to claim 15 wherein the metal dispersed on the support material in the first zone is palladium, and the absorbent has a palladium content in a range of from about 0.01 to about 10 percent based on the total weight of the adsorbent.

17. The process according to claim 10 wherein the olefin in the gaseous mixture being purified is predominantly ethylene or propylene, wherein the adsorbent in the first zone has a metal dispersion value in a range upward from about 20 percent to about 80 percent as measured by carbon monoxide chemisorption method, and wherein the gaseous mixture, while passing through the bed in the first zone, is at temperatures in a range of from about 35° C. to about 65° C.

18. A process for purification of a gaseous mixture to obtain a feedstock suitable for formation of polymer using a metallocene catalyst system, which purification process comprises:
providing an impure gaseous mixture comprising at least about 99 percent by volume of ethylene, and acetylene in an amount in a range upward from about 1 to about 1000 parts per million by volume based upon the total amount of ethylene present and optionally saturated hydrocarbon gases;
passing the impure mixture through a first zone containing a bed of adsorbent which has retained a substantial amount of carbon monoxide, the adsorbent comprising at least about 90 weight percent of gamma alumina having surface area in a range of from about 150 to about 350 square meters per gram as measured by the BET gas adsorption method, on which is dispersed palladium in the zero valent state, to effect, under conditions suitable for adsorption within the bed, selective adsorption and/or complexing of the contained acetylene contaminant with the adsorbent, and thereby obtain an effluent mixture which contains less than about 1 part per million by volume of acetylene and an amount of carbon monoxide in a range upward from about 10 parts per million by volume;
passing the effluent mixture through a second zone containing a bed of adsorbent which comprises at least one metallic element in a positive valent state selected from the group consisting of calcium, titanium, vanadium, chromium, manganese, iron, cobalt, nickel, copper, zinc, zirconium, molybdenum, palladium, silver and tin, to effect, under conditions suitable for adsorption within the zone, selective adsorption and/or complexing of the contained carbon monoxide with the adsorbent therein, thereby obtain a feedstock which contains less than about 5 parts per million by volume of carbon monoxide and less than about 1 parts per million by volume of acetylene;
effecting, in the presence of an essentially dihydrogen-free atmosphere within the bed in the first zone, selective adsorption and/or complexing of the contained acetylene with the adsorbent, until levels of acetylene in the effluent mixture increase to a limiting level in a range downward from about 1 parts per million by volume; and
thereafter regenerating the resulting bed of adsorbent in the first zone in the presence of a reducing gas comprising dihydrogen and containing at least 50 parts per million of carbon monoxide, to effect release of the contained acetylene from the adsorbent.

19. The process according to claim 18 wherein the adsorbent in the second zone comprises copper(I).

20. The process according to claim 19 wherein the adsorbent in the first zone contains less than 500 parts per million by weight of a sulfur-containing component, calculated as elemental sulfur, and the absorbent in the first zone has a palladium content in a range of from about 0.01 to about 10 percent based on the total weight of the adsorbent.
